(19) United States
(12) Patent Application Publication
(10) Pub. No.: US 2025/0176963 A1
(43) Pub. Date: Jun. 5, 2025

(54) Nerve Wrap Using Constructed Tissue

(71) Applicant: Vascudyne, Inc., St. Paul, MN (US)

(72) Inventors: Zeeshan Syedain, St. Paul, MN (US); Richard F. Murphy, White Bear Township, MN (US)

(21) Appl. No.: 18/845,665

(22) PCT Filed: Mar. 10, 2023
(86) PCT No.: PCT/US2023/015002

§ 371 Date: Sep. 10, 2024

Related U.S. Application Data
(60) Provisional application No. 63/318,767, filed on Mar. 10, 2022.

(51) Int. Cl. A61B 17/11; A61L 27/22; A61L 27/38

(52) U.S. Cl. CPC A61B 17/1128; A61L 27/225; A61L 27/38; A61B 2017/1132; A61L 2430/32

(57) ABSTRACT

The present invention is constructed or engineered tissue, suitable for isolating, protecting, repairing, and/or regenerating a nerve or a portion thereof. The invention includes a nerve wrap, enclosure, conduit, or tubular shape formed from the tissue, methods of making the nerve wrap, methods of using the wrap, and methods of treatment using the wrap. The invention also provides a product for treating other types of tissue damage.

### FIELD OF THE INVENTION

The invention relates to products formed from proprietary regenerative tissue, nerve wrap products, an implant comprising this tissue, and methods of treating nerve conditions and/or disorders using such tissue.

### I. BACKGROUND OF THE INVENTION

A relatively new field of medicine—since the early 1990s—is the field of Regenerative Medicine. Regenerative Medicine is the process of creating functional tissues to repair, replace, or restore tissue or organ structure and function lost due to age, disease, damage, or congenital defects. This field of medicine uses new methods and products including (stem) cell therapy, development of medical devices, and tissue engineering.

The use of prepared heterogenous graft material for human surgical implantation is well known. More specifically, the use of treated animal tissue as human tissue grafts, replacement valves, and similar implantation surgical procedures is well known. However, problems of immunogenicity, thrombogenicity, calcification, material strength, and size have not been adequately addressed in the prior art.

Since the 1930's, medical researchers have attempted to develop suitable natural and synthetic alternatives for obtaining small diameter grafts useful in vascular surgery. Historically, attempts to fabricate such tubular grafts from man-made materials have been somewhat unsuccessful. Homologous tissues are not always readily available and are not always readily available in the size the surgeon needs. Furthermore, some of these tissues may be immunogenic and therefore may require processing or certain treatments to reduce their immunogenicity.

Chronic nerve compression remains one of the most common forms of peripheral nerve injury. For example, carpal tunnel syndrome (CTS) incidence may be 10% of the general population (2-3) and leads to 400,000-500,000 carpal tunnel release procedures each year in the US. While carpal tunnel release relieves the symptoms associated with CTS, a significant number of patients do not experience satisfactory results. Poor outcomes are linked to scar tissue formation within and/or around the nerve and to the formation of adhesions between the nerve and surrounding tissues (5). Native tissues such as adipose tissue (6) and vein (7) have been used as protective barriers in CTS surgery. There are however shortcomings associated with the use of autologous tissue including donor site morbidity, limited availability of healthy autologous tissue and the risk of surgical complications. A variety of materials are available, but all have shortcomings, and none are regenerative. There remains a need for a solution that combines the benefits of native collagenous tissue having regenerative properties with those of manufacturing at scale and in the proper form (i.e., wrap and/or conduit).

Moreover, the performance of the tissue described below in demanding vascular applications is evidence of suitability for neural applications and a natural extension of the development to serve the neurosurgical market, reduce healthcare costs and improve patient recovery and outcomes.

Notwithstanding the usefulness of the above-described methods, a need still exists for increasing patency; making an implant less thrombotic in structure and/or function; minimizing calcification; and increasing the useful life of the implant.

### II. SUMMARY OF THE INVENTION

One embodiment of the invention is the preparation and use of constructed tissue (CT) to make implants, their use as implants, and their use in mediating nerve treatment or therapy.

An advantage of both embodiments of the invention is the starting material itself.

The products, uses, and processes of the present invention are suitable for treating diseases and conditions that would benefit from regenerative engineered tissues, especially those involving tubular tissue constructs. One such use is for nerve protection, repair, and regeneration.

An embodiment of the invention includes a tubular engineered tissue device with characteristics suitable for use as a peripheral nerve protection device (e.g., dry/sterile) or a regenerative engineered tissue peripheral nerve wrap.

Some embodiments of the invention include the manufacture of a graft that is durable and has been demonstrated in both animal and human trials to withstand the rigorous mechanical requirements of the vascular system (8). This suggests that they provide adequate support to nerves of a similar diameter where their primary function is that of a barrier between the healing nerve and surrounding tissue.

The biological materials according to the present invention, are processed to modify (e.g., reduce or eliminate) calcification characteristics, resorbability, size and shape, thinness, collagen content, and other characteristics and properties that will become clear from the description of the invention. The methods, uses, and products of the present invention are intended for implant in a mammal, preferably a human. All of the biological materials, processed according to the present invention, are appropriate for use in an in vivo environment, and include one or more of the following desirable properties for graft material suitable for implantation: a) size compatibility with surrounding vessels to which it will be anastomosed; b) sutureability, kink resistance, softness, radial and longitudinal compliance, and flexibility (a softer hand); c) non-thrombogenicity or low levels of thrombogenicity; d) durability; e) ease of sterilization; f) readily available, and available in diameters and lengths appropriate for surgical procedures; g) shelf life appropriate for market conditions (typically greater than three years); h) resistant to infection; i) sufficient strength to resist aneurysm formation; j) non-immunogenic; k) resistant to degradation; I) resistant to formation of neointimal hyperplasia; and m) tactile, as expressed by surgeons using the tissue and/or grafts of the present invention, particularly suitable for microsurgery.

Surgeons have also expressed superior quality of the tissue of the present invention in terms a tactile response, specifically in comparison to Axogen products and typical vasculature (usually autologous, e.g., venous grafts during coronary artery bypass procedures).

Although specific advantages have been enumerated above, various embodiments may include some, none, or all of the enumerated advantages.

Other technical advantages may become readily apparent to one of ordinary skill in the art after review of the following figures and description.

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

With the following enabling description of the drawings, the apparatus should become evident to a person of ordinary skill in the art.

### III. BRIEF DESCRIPTION OF THE FIGURES

FIG. 1 shows a device of the present invention in place in vivo.

FIG. 2 shows a medical procedure for protecting a sciatic nerve. FIG. 2A, the nerve is isolated; FIG. 2B the nerve is cut; FIG. 2C, the nerve ends are reattached.

FIG. 3 shows an in-situ comparison of a tissue of the present invention (3A) compared to a commercially available protector (3B).

FIG. 4 shows the 30-day results of the experimental comparison described in Example 3.

FIG. 5 shows the 30-day histological results of the experimental comparison described in Example 3.

### IV. DETAILED DESCRIPTION OF THE INVENTION

The present invention is a product and process for use in nerve isolation, protection, repair, and regeneration. The product is a nerve graft, wrap, or tube that is customizable and designed to bridge nerve gaps, permitting restoring continuity to nerve function, nerve isolation, nerve protection, and/or nerve repair or regeneration.

As used herein, nerve refers to human or animal fibers that send impulse(s) to and from the central nervous system. By way of non-limiting examples, nerve includes but is not limited to a portion of a nerve, e.g., an axon or axon extension; human or animal sensory neurons; and defined nerves or axons, including but not limited to CNS axons, peripheral nerves. The invention also includes any human or animal tissue to which the products of the present invention can be applied. E.g., enclosed or wrapped by a product. Without intending to be limited thereby, a partial list of tissues includes vascular tissue, urological tissue, tendons, or muscle tissue.

As used herein, isolation refers to isolating the nerve from the surrounding environment, i.e., isolate injured nerve within invention to prevent nerve axons migrating away from native nerve, isolate nerve from surrounding inflammatory cells.

As used herein, protection refers to protecting from surrounding environment including inflammatory cells, fibrotic scars, mechanical and chemical signals that can act on nerve.

As used herein, repair refers to isolation and protection of the nerve by the tissue mediating natural repair. In another embodiment, repair refers to isolation and protection of the nerve by tissue to repair, wherein the tissue comprises one or more active agent mediating repair.

As used herein, regeneration refers to isolation and protection of the nerve by the tissue mediating natural repair. In another embodiment, as used herein, repair refers to isolation and protection of nerve by tissue to regeneration, where the tissue comprises one or more active agent mediating regeneration.

In some embodiments, the nerve ends are aligned and encapsulated, enclosed, or wrapped in the tissue of the present invention. In some embodiments, the nerve ends are enclosed within a tubular structure or positioned with a tube formed from a tissue of the present invention.

A tube or tubular material of the present invention may be positioned to surround, contain, or enclose a nerve, nerve end, or a portion of a nerve in configurations or dimensions as required or desired. In some embodiments, the tubular material may include one or more seams, wherein the seams need to be overlapped or closed (a sheet rolled into a tube or edges of a sheet or tube joined and sealed). For example, overlapping tissue (or partially overlapping) may be rolled into a tubular construct. In some embodiments, the tubular construct or wrap can be sized to accommodate different nerve sizes and diameters.

The graft or wrap of the present invention may be used in a method of treating a patient having nerve damage, including but not limited to peripheral or CNS nerve discontinuities. In some embodiments, the graft or wrap supports axonal regeneration across a nerve gap.

The present invention also includes methods of treating injuries to nervous system tissue. Some embodiments include contacting, wrapping, or enclosing a nerve or a portion thereof, wherein the nerve area is an area of injury, disease, or intentional severing (e.g., a nerve end or one or both ends of a nerve gap). Examples include but are not limited to one or more nerves of the CNS (e.g., cranial nerve or spinal nerve), or a peripheral nerve. Examples of nerves are very well known in the art.

The present invention also includes methods of treating nerve conditions or injuries wherein a gap exists between a proximal end and a distal end of the nerve. These methods include introducing a tube or tubular structure of the present invention (i.e., comprised of the tissue of the present invention) into the gap, wherein one end of the tubular structure encloses the proximal end, and a second end of the tubular structure encloses the distal end of the nerve.

An embodiment of the invention includes but is not limited to treating patients with nerve damage or condition, including but not limited to cut, lacerated, compression, stretched, and neuroma. Exemplary damage or conditions include but not limited Brachial plexus or “burner” neck/shoulder nerve injury, Radial nerve injury, Carpal tunnel syndrome, Ulnar elbow entrapment, bicycler's neuropathy, Ulnar wrist entrapment. Some embodiments may include treating trauma injuries; trauma from accidents; surgical reconnection of disconnected tissue; transplantations in order to avoid phantom pain; preservation of wound growth areas; protectors and incubators for bedsore areas (and other long term damaged areas, particularly wound areas that threaten or encroach on open nerve areas.

In some embodiments, the invention further includes combining the graft with one or more active agents, e.g., an NSAID, a growth factor, an immunosuppressive agent, a regenerative agent (e.g., a laminin), or the like.

In another embodiment of the invention, the tissue may include structures or agents (e.g., active agents) within the surface of the CT tissue, e.g., to promote directional nerve regeneration. For example, for a lacerated nerve with a gap, the gap between lacerated nerve end maybe filled and covered by the CT of present invention, allowing nerve to reconnect within the cover.

The tissue of the present invention may be any size or shape. In a preferred embodiment, the tissue is the form of a wrap, conduit, cover or tube. Nerve wrap shall be used in describing the invention(s) below, but it is intended that the invention should not be limited by this short-hand description.

The present invention is a graft, prosthesis, or covering formed from constructed (CT) regenerative, and/or engineered tissue. As used herein, constructed or engineered refers to the fact that the inventors and others may produce or construct the tissue, e.g., the tissue is not a product of nature. The invention includes methods of making the tissue and methods of making the graft or prosthesis. In accordance with the present invention, the tissue may be regenerative or not regenerative and/or the tissue may induce recellularization or not. Embodiments of the invention also may include placing a material of the present invention into a cavity or gap in a nerve (e.g., a severed end of a nerve), and enclosing or covering each end of the nerve with an end of the material.

Embodiments of the invention may also include protecting the nerve ends by covering them or wrapping them with a tissue product of the present invention, thereby allowing the nerve ends to heal or grow in a localized environment that is isolated from an area away from the nerve ends. In these embodiments, isolating and protecting the nerve ends may prevent or reduce neuroma formation, scar tissue formation, aberrant nerve growth, and the like.

In preferred embodiments, the nerve wrap of the present invention exhibits reduced, not detectable, or no inflammation or immune response.

The tissue may be formed by combining ECM-producing cells in the presence of fibrinogen and thrombin under conditions that permit the formation of regenerative tissue. The tissue is then cultured until it matures, e.g., is substantial enough to be used for its intended purpose. Some embodiments of the tissue have been previously described. For example, see the patents and patent applications listed below.

The tissue wrap of present invention maybe delivered in any medically acceptable manner. In a preferred embodiment, the tissue is surgically delivered. In another embodiment, the tissue is delivered via catheter or tube.

A preferred embodiment of the invention is any tubular structure formed from the tissue of the present invention, including but not limited to a tubular graft.

In accordance with embodiments of the present invention, any prosthesis may be formed in whole or in part using regenerative tissue (RT) or engineered tissue. RT, as used herein, refers to tissue formed or processed as disclosed in the following: 2007/061800; WO 2007/092902; 2016/0203262; WO/2004/018008; WO 2004/101012; PCT/US21/62709 (filed 9 Dec. 2021); PCT/US2017/026204 (filed 5 Apr. 2017); U.S. Pat. Nos. 10,111,740; 10,105,208; 10,893,928; 8,192,981; 8,399,243; 8,617,237; 8,636,793; 9,034,333; 9,126,199; U.S. Ser. No. 17/139,575 filed Dec. 31, 2020 (issue fee paid); U.S. Ser. No. 16/500,147 filed Oct. 2, 2019 (issue fee paid); U.S. Ser. No. 10/523,618; U.S. Ser. No. 10/556,959; U.S. Ser. No. 13/771,676; 2015/0012083; 2009/0319003; 2011/0020271; 2012/0230950; 2013/0013083; 2014/0330377; 2014/035805; 2017/0135805; 2017/0296323; 2017/0306292; U.S. Pat. Nos. 8,198,245; 9,127,242; 9,556,414; 9,657,265; and 9,650,603; all of which are hereby incorporated in the entirety be reference.

In one embodiment of the invention, the bioengineered tissue may be made according to U.S. Pat. Nos. 10,111,740; 10,105,208; and 10,893,928, all Tranquillo, et al., each incorporated in its entirety be reference. Any process or method for producing engineered tissue is included within the scope of the present invention.

The CT of the present invention, may be characterized by modified (e.g., reduce or eliminate) inflammation, calcification characteristics, resorbability, suture retention, size and shape, thinness (e.g. dilatation or aneurysm formation), collagen content, and other characteristics and properties that will become clear from the description of the invention.

The methods, uses, and products of the present invention are intended for implant in a mammal, preferably but not limited to a human.

The CT of the present invention is distinct from certain other kinds of regenerative or engineered tissue in the use of crosslinked fibrinogen that is later degraded during the culturing process. Also, the CT of the present invention can be contracted or allowed to contract, for example, in the longitudinal direction and/or in the radial direction, among others. In accordance with some embodiments of the invention, the fibers in the tissue may align or become aligned, believed to be partially due to fibrin having no or little resistance to contraction that occurs naturally as part of the collagen/ECM formation process. The inventors also believe that radial and/or longitudinal contraction occurs in part naturally as an inherent function of tissue forming as described herein. In another embodiment of the invention, the contraction may be scalable or intentionally controlled to enhance, promote, or achieve one or more tissue characteristics, e.g., fiber alignment, or tensile strength, or suturability. Furthermore, the CT of the present invention does not include any synthetic materials, as is typical in other processes that use PLA or PGA or the like.

A product and/or method of the present invention typically includes combining fibrinogen or fibrinogen-like material, thrombin, and matrix-producing cells to produce a fibrin gel with a homogeneous cell suspension. In preferred embodiments of the invention, the cell infused fibrin gel undergoes casting, used herein to refer to encapsulating cells in a fibrin gel, and culturing to form the collagenous tissue or grafts. In other preferred embodiments, the tissue or graft may be allowed to contract (e.g. longitudinally or radially), preferably in a controlled manner. In accordance with the present invention, the process permits customized or optimized fiber alignment during the contraction phase. Customized or optimized alignment includes, but is not limited to radial alignment, longitudinal alignment, both radial and longitudinal alignment, and a pre-determined ratio of radial and longitudinal alignment.

One or more methods of the present invention may also include molding or forming the cell-seeded fibrin gel into a pre-determined shape; manipulating, mechanically and/or manually, the tissue in the presence of culture medium to produce CT; manipulating the tissue during the culturing phase of the tissue; manipulating the tissue during the maturation phase of the tissue; manipulating the tissue during the culturing/maturation phase of the tissue production process; manually moving the graft to evenly distribute the stress relief from the contracting ends; decellularizing the CT; and automated or semi-automated versions of any of the method steps.

Each of these ingredients and steps will now be described in more detail.

The CT of the present invention may be characterized by one or more of the following: non-oriented fibers; oriented fibers; thickness up to about 1 mm, preferably between about 100 μm and about 500 μm; non-immunogenic or minimally immunogenic; a tissue, sheet or shape that is anisotropic; a tissue, sheet, or shaped structure produced by a process that includes scaled contraction (as described above); a sheet or shape that is suitable for cutting into shapes, e.g., by scalpel, die, or laser; suppleness; suturability; no or little calcification during life of implant; crosslink density, or variations of crosslink density through the material thickness; collagen concentration; collagen density, or variation of crosslink density through the material thickness; remodeling proclivity; absorption; resorption; degradability, regions of greater stiffness; and regions of greater flexibility.

In preferred embodiments of the invention, the RT has oriented fibers leading to suture pull-out resistance, anisotropic material properties as witnessed by tensile strength, even more preferably, adapted for its end use (e.g., a sheet, or a tube, or a valve).

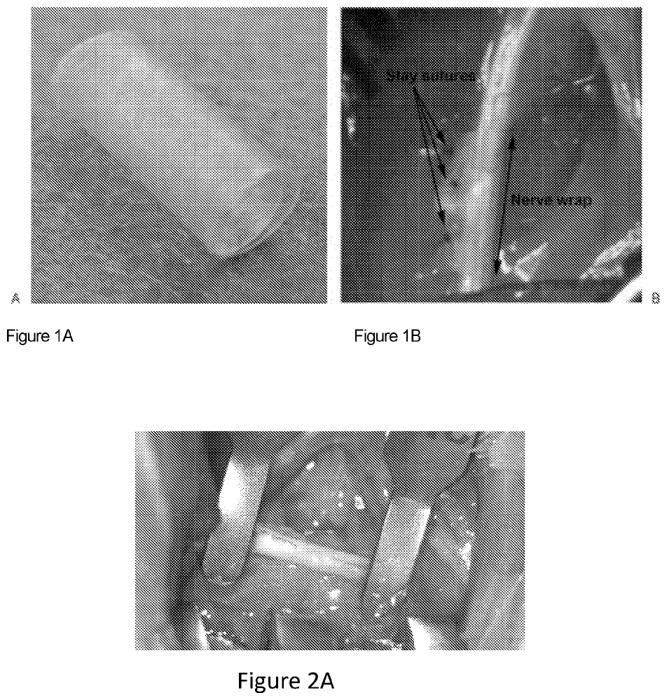

Referring now to the figures, FIG. 1 shows an exemplary wrap or tubular structure of the present invention. The illustrated wrap is sized and shaped as a peripheral nerve wrap. FIG. 1A shows a side view, also illustrating an overlapping portion of the wrap, which, in an open position can be easily slipped around the nerve; and in a closed position (illustrated), can enclose or close around the nerve. FIG. 1B shows the wrap of FIG. 1A in place as a barrier between a dissected sciatic nerve and the surrounding tissue bed.

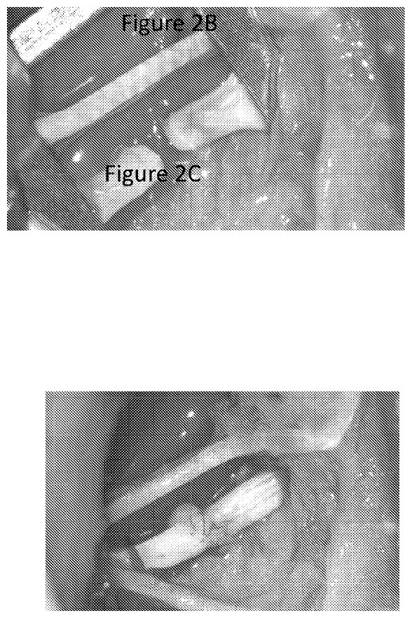

FIG. 2 illustrates a surgical operation. FIG. 2A shows isolating a sciatic nerve. FIG. 2B shows a sciatic nerve dissection or gap. FIG. 2C shows the sciatic nerve reattached with a 9-0 prolene suture.

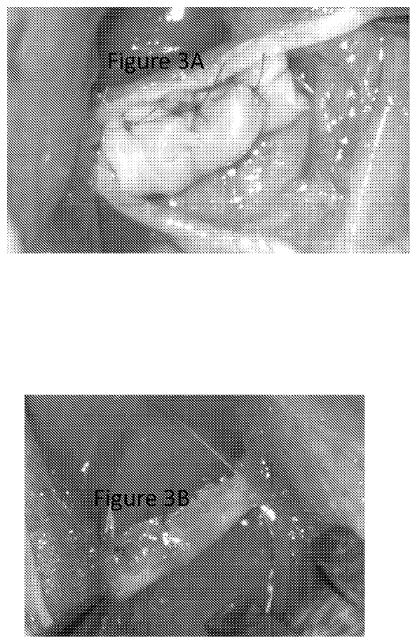

FIG. 3A shows the end of the operation of FIG. 2, illustrating the sciatic nerve shown in FIG. 2 wrapped and protected by the wrap or enclosure of FIG. 1.

FIG. 3B shows a sciatic nerve enclosed by a commercially available product (Axogen Nerve Protector).

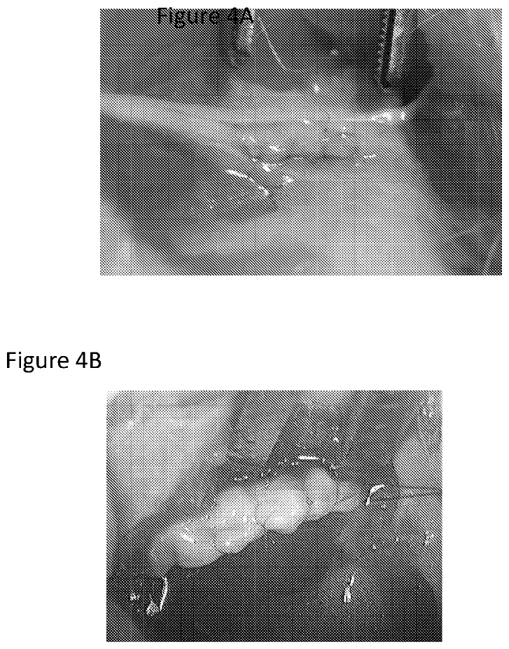

FIG. 4 shows the results of the comparison experiment described in Example 3. FIG. 4A shows that scar tissue develops around the commercially available wrap. FIG. 4B shows a wrap of the present invention at day 0 (implant day); FIG. 4C shows the wrap at day 30. FIG. 4B is provided to show that the wrap can have slack and wrinkles when implanted. FIG. 4C shows that no scar tissue develops after 30 days, and that the wrap of the present invention remodels into a smooth texture.

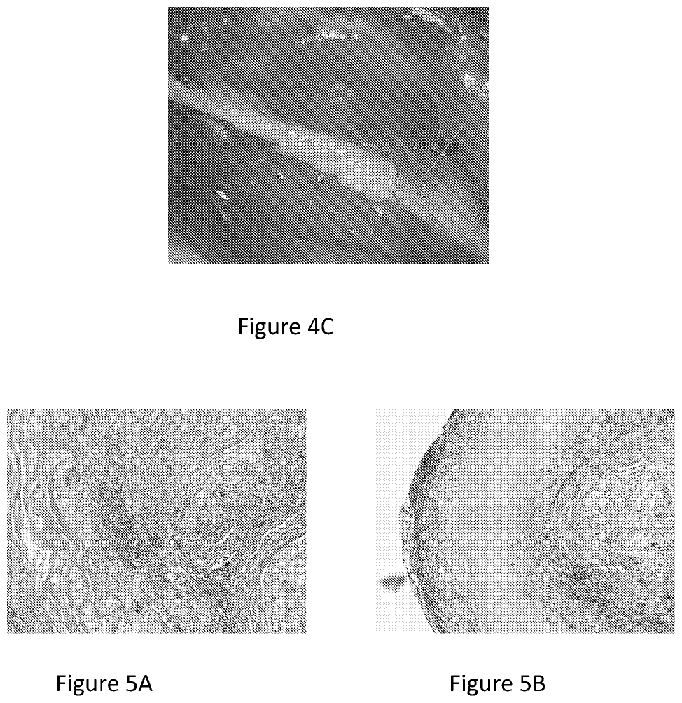

FIG. 5 shows the histological comparison of the comparison experiment in FIG. 4. FIG. 4A shows the commercially available wrap at 30 days and shows signs of immune response and scar tissue. FIG. 5B shows the wrap of the present invention, showing the lack of an immune response, lack of any scar tissue, evidence of new vascular growth around the nerve, and evidence of interstitial cell formation within the matrix.

It should be understood that the tubular product, tube, and wrap of the present invention may be used for repairing or treating other types of nerve damage and other types of tissue other than nerves. For example, the tissue or a product of the present invention may be used to treat or repair gap repairs (e.g., providing a covering, conduit, or enclosure between the ends of a transected nerve). The tissue or a product of the present invention may be used to treat or repair other types of tissues including but not limited to muscle, tendon, organs (e.g., kidneys and liver) skin, vasculature, bladder, fascia, and uterus.

### Various Method Steps:

By mimicking the extra cellular matrix of the natural environment, a tissue can be grown having good structural properties, which eventually develop towards a native-like architecture (i.e., the tissues of the present invention are a biomimetic material).

Some embodiments of the invention may further include storing and/or sterilizing a medical device or tissue of the present invention. These embodiments may include preselected storage solution; preselected sterilization solution or technique; storage packaging; and/or sterilization packaging. In one embodiment, the tissue may be stored in PBS and refrigerated until use. In another embodiment, the tissue may be partially or fully dehydrated. In one embodiment, the storage is in a sterile dry container. Other storage/sterilization processes may include one or more additives known to those with skill in the art. In another embodiment, the tissue may be E-beam sterilized in PBS alone.

One skilled in the art will recognize that other storage and sterilization protocols may be used with the tissue of the present invention.

Some embodiments of the invention include a method for promoting or mediating nerve cell growth between a first nerve end and a second nerve end. These methods may include one or more of the following: introducing one or more wraps or conduits around one or more nerve ends; providing one or more nerve growth agents into the cavity between the nerve ends or in the lumen of the conduit; forming a fluid or factor communication between a first nerve end and a second nerve end; filling the space between a first nerve end and a second nerve end with an active agent; and introducing a growth and/or repair factor or active agent into the space between and/or around one or more nerve ends. In preferred embodiments, the mediator agents may be included or positioned in the cavity or lumen of a wrap, tube, or tubular structure of the present invention.

### Other Embodiments

The products and methods of the present invention can be used to produce nerve wraps of pre-determined or desirable dimensions, including length, perimeter, width of the tissue, circumference, and cross-sectional dimension. For example, a typical wrap may have a length of about 3 mm to about 200 mm, including any length in between. A wrap of the present invention may also define or enclose a total volume from about 3 mm³ to about 65,000 mm³.

As noted above, a wrap or product of the present invention may include one or more active agents, including but not limited a growth factor, a growth suppressor, an immunosuppressive agent, a nerve regeneration promoter, and an anti-inflammatory agent. Other exemplary active agents include but are not limited to glial cell-derived neurotrophic factor, axonal growth and elongation factors, Schwann cell growth factors, ciliary growth factor, nerve growth factor, and the like. These active agents are known to those skilled in the art.

The tissue of the present invention may include one or more collagens, including but not limited to collagen type I, II, III, IV, and V.

The wrap of the present invention may carry, hold, deliver, be coated with, or be infused with a variety of cells (or more than one cell type) and other active agents (defined above). Exemplary cells include but are not limited to Schwann cells, macrophages, and other cells that promote or mediate nerve growth, repair, or regeneration.

A wrap or conduit of the present invention may be used to create or establish a protected or isolated microenvironment around a nerve or nerve ends. This enclosure permits localized concentrations of neurogenic and trophic factors, protects against scar tissue formation, blocks escaping axons, and promotes directional axon growth.

As noted above, a tissue or product of the present invention may be used to protect or repair other types of tissues or repairs. Exemplary tissues include but are not limited to muscle, tendons, vasculature, skin, fascia, or organs. Exemplary repairs include but are not limited to hernia repair, patch tissue defects, or tendon repairs (e.g., rotator cuff), and may used to repair or treat injuries or damage involving two or more different tissue types, e.g., tendon to bone or ligament to bone.

A tissue product of the present invention be unsupported or may further include a support member, such as a nitinol stent or scaffold. The support member many be internal, external, or embedded in the tissue.

A tissue product or wrap of the present invention is flexible and capable of manipulation so that the product or tissue can be bent or wrapped around a nerve or other tissue, without breaking or damaging the tissue in need of repair. A wrap of the present invention may also be flexible enough to wrap the material around a nerve, or to be pulled, pushed, crimped, or manipulated around a nerve or other tissue.

As noted above, a wrap or tissue product of the present invention is a tube or conduit, or tubular is shape. A preferred embodiment is a continuous tubular shape. Other preferred embodiments are shown in the Figures. Typically, these shapes include or enclose a lumen through the longitudinal length of the construct. The lumen can be open or closed at one or both ends.

In some embodiments of the invention, the wrap or conduit can provide a temporary or removable cover, cap, or wrap around a nerve or other tissue. In these embodiments, the product is intended to provide temporary repair or protection, preferably prior to a more permanent or long-lasting repair or treatment.

In some embodiments, the wrap, tube, or tubular structure may include a a slit within a wall of the structure, such as a longitudinal slit. An exemplary view of this feature is shown in FIG. 1.

In some embodiments of the invention, the wrap, tube, or tubular structure creates a nerve conduit or enclosure for treating or protecting a nerve of one or more nerve ends. In these embodiments, the product of the present invention defines a space or cavity around a nerve or between one or more nerve ends.

Preferred embodiments of the invention include a medical device or prosthesis of the present invention packaged and ready to use by the surgeon or in the operating room.

Some embodiments of the invention may include a scaffold or stent, optionally degradable, that can be seeded by extracellular matrix producing cells. The scaffold may be formed from fibrin, PLA, PGA, or other synthetic or biological polymer, and mixtures thereof. The ECM producing cells can be cultured with the scaffold, allowing the cells to produce ECM, which can in turn replace the degradable scaffold. Optionally, the scaffold can be manipulated or processed (as described herein) to create alignment of the fibers in the ECM (e.g., an anisotropic matrix). The final product, preferably in the form of a sheet, may be decellularized using detergents, or dehydrated (e.g., freeze drying), to create a sheet of engineered tissue with or without cells.

The present invention also is a surgical kit comprising one or more of the following: a regenerative tissue implant or graft processed or produced according to the present invention; one or more instruments for implanting the graft; a rinse tray; a rinse solution, e.g., heparin; and suture material.

One of the embodiments of the present invention is a sterile closed package containing a biological material of the present invention. Typically, a separate container would hold individual or multiple samples having known size or dimensions. If desired, the biological material in the sterile package can be attached to another material or structure, such as an annuplasty ring, a sewing cuff, a synthetic graft, or a support for positioning the biological material on a stapler.

In accordance with preferred embodiments of the invention, pre-determined shape refers to any forming or shaping the natural tissue into any shape or form that mediates tissue reaction when it is implanted.

The present invention also includes tubular grafts in a variety of diameters and lengths as needed to match the anatomy of the commonly injured nerves (e.g., brachial plexus, radial nerve, ulnar nerve, median nerve) (9).

This invention represents a series of related products addressing increasingly complex needs in the nerve space (i.e., nerve wrap, nerve conduit for nerve gap,)

In first embodiment of the invention-Nerve Wrap to protect injured nerves from chronic inflammation and adhesion formation following compression injury (e.g., carpal tunnel syndrome) where current solutions have shortcomings.

In second embodiment of the invention—A nerve conduit to protect the proximal and distal ends of transected nerve, mediate nerve therapy in a protective environment over relatively short gaps (<5 cm) where current solutions have shortcomings.

In third embodiment of the invention—A nerve graft combining a tissue conduit with one or more active agent to mediate nerve therapy over long gaps (>5 cm).

One skilled in the art will recognize that the processes steps described herein may be variously modified and a wide variety of ways in order to achieve a tissue with certain properties, according to present invention.

### Definitions

The following definitions are used in reference to the invention:

As indicated herein, a decellularized vessel consists essentially of the extracellular matrix (ECM) components of the vascular tree. ECM components can include any or all of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei in histologic sections using standard histological staining procedures. Preferably, but not necessarily, residual cell debris also has been removed from the decellularized organ or tissue.

(B) As used herein, biomimetics or biomimicry refer to imitating the models, systems, and elements of nature for the purpose of solving complex human or animal problems. In the present invention, biomimetics is used for therapeutic purposes.

### REFERENCES

1) Global Nerve Repair Biomaterials Market-Industry Trends and Forecast to 2028. Data Bridge Market Research, 2019.
2) de Krom M C, Knipschild P G, Kester A D, Thijs C T, Boekkooi P F, Spaans F. Carpal tunnel syndrome: prevalence in the general population. J Clin Epidemiol 1992; 45:373-375.
3) Nordstrom D L, DeStefano F, Vierkant R A, Lyde P M. Incidence of diagnosed carpal tunnel syndrome in a general population. Epidemiology 1998; 9:342-345.
4) Muller M, Tsui D, Schnurr R, Biddulph-Deisroth L, Hard J, MacDermid J C. Effectiveness of hand therapy interventions in primary management of carpal tunnel syndrome: a systematic review. J Hand Ther 2004; 17:210-228.
5) Stutz N, Gohritz A, van Schoonhoven J, Lanz U. Revision surgery after carpal tunnel release-analysis of the pathology in 200 cases during a 2-year period. J Hand Surg. 2006; 31:68-71.
6) Thakker A, Sharma S C, Hussain N M, Devani P, Lahiri A. Nerve wrapping for recurrent compression neuropathy: A systematic review. J Plast Reconstr Aesthet Surg. 2021; 84 (3): 549-559.
7) Sabongi R G, Fernandes M, Goes dos Santos J B. Peripheral nerve regeneration with conduits: use of vein tubes. Neural Regeneration Research 2015; 10 (4).
8) Syedain Z H, Graham M L, Dunn T B, O'Brien T, Johnson S L, Schumacher R J and Tranquillo R T. A completely biological “off-the-shelf” arteriovenous graft that recellularizes in baboons. Sci Transl Med. 2017; 9.
9) Bermeister K D, Grobe-Harlage L, Daeschler S C, Rhodius P, Boker A., Beyersdorff M. Acute and long-term costs of 268 peripheral nerve injuries in the upper extremity. https://journals.plos.org/plosone/article?id=10.1371/journal.pone.0229530
10) Kokkalis Z T, Pu, C, Small G A, Weiser R W, Venouziou A I and Sotereanos D G. Assessment of Processed Porcine Extracellular Matix as a Protective Barrier in a Rabbit Nerve Wrap Model. J Reconstr Microsurg 2011; 27 (1): 019-028.

### EXAMPLES

### Example 1

Nerve Wrap Process Development: The current process is used to manufacture devices suitable for use as a nerve isolation and or protector. Some modifications/extensions to the existing process may be required. These alternative or variations include changing lumen diameter, thickness of tissue, final product texture via drying, packaging and sterilizing the devices prior to implantation. One or more of these alternatives may result in greater “ease of use” of the product or be more appropriate, particularly in human use.

To evaluate nerve protection with engineered tissue wrap, a study in a well-established rat model was used to compare engineered tissue with commercially available nerve wrap protector. The animal model and study design are based on work by Zhukauskas et al. (10).

Surgery was performed on 12 animals (Sprague Dawlet rat). Surgery involved transecting sciatic nerve by cutting with scissor then reattach using 9-0 prolene sutures. The reattached sciatic nerve is wrapped with a tubular product as exemplified in FIG. 3A or Predicate CONTROL (e.g., FIG. 3B. The wrap was trimmed to the appropriate size, then laid under the nerve, rolled around the nerve, then connected using 8-0 prolene sutures. Treatment was randomized among 12 animals, with each animal having one leg sciatic nerve treated with CT or Commercial predicate device (porcine SIS tissue wrap, commercially available from Axogen).

### Study Chronology:

Day 0:12 animals undergo surgery; all were treated same way for transection and reattachment of the sciatic nerve. Randomly, 6 animals had nerve wrapped with CT and other 6 with predicate device. Three animals for each wrap type are assigned to one of two groups (Group 1, or 2).

Day 30 (1 month): 6 animals in Group 1 are euthanized, the sciatic nerves are exposed, imaged, and adhesions are assessed. The nerve segments (TREATMENT and Predicate CONTROL) are harvested for histopathologic analysis.

Day 90 (3 months): 6 animals in Group 3 are euthanized, the sciatic nerves are exposed, imaged, and adhesions are assessed. The nerve segments (TREATMENT and Predicate CONTROL) are harvested for histopathologic analysis.

### Study Outcomes:

Following surgery, all animals recovered without any adverse events. All animals showed mobility and movement in treated leg. In all cases, there was curling of toes, at both Day 30 and Day 90. At Day 30 and Day 60, exposed treated site showed evidence of scar tissue and fibrotic adhesion around the Predicate CONTROL, however no fibrotic adhesion was seen around the CT wrap. Compared to the implant, the CT wrap had smooth texture indicating cellular remodeling, as wrinkles formed during surgical implant were no longer observed in any of the TREAMENT CT. The nerve with wrap were cut out, imaged and cut into smaller samples for cross-section histology analysis. Explant Sections were processed for histology and slides stained with H&R and Trichrome. H&E (Hematoxylin & Eosin) stain showed presence of macrophage in and around the Predicate Control, while no such macrophage presence was seen in and around CT. Further there was presence of invading cells into CT form both external and internal surface of the wrap.

### Example 2

Nerve Connector Process Development: The current process was used to manufacture devices suitable for use as a nerve connector for transected nerve with a gap. Some modifications/extensions to the existing process may be required or beneficial. These include changing lumen diameter, thickness of tissue, final product texture via drying, packaging and sterilizing the devices prior to implantation. Some of these variations or alternatives are more appropriate or desirable for greater “ease of use” of the product, particularly in human use.

To evaluate nerve connection with engineered tissue wrap, a study in a well-established rat model was used to compare engineered tissue with commercially available nerve wrap protector. The animal model and study design are based on work by Zhukauskas et al. (10).

Surgery was performed on 12 animals (Sprague Dawlet rat). Surgery involved transecting sciatic nerve by cutting with scissor, then a gap of 5 mm is established between proximal and distal nerve ends. The CT tissue connector in a form of a tube is then placed around each exposed end of the nerve at proximal and distal end. Using 8-0 prolene, two suture knots are placed on each end, securing the connector in place. Saline is injecting into the connector to fill with fluid, prior to closing the surgical size. Treatment will be randomized among 12 animals with each animal having one leg sciatic nerve treated with CT or Commercial predicate device (porcine SIS tissue connector, commercially available from Axogen).

### Study Chronology:

Day 0:12 animals undergo surgery; all were treated same way for transection and establishing a 5 mm gap in the sciatic nerve. Randomly, 6 animals had nerve connector with CT and other 6 with a predicate connector. Three animals for each wrap type are assigned to one of two groups (Group 1, or 2).

Day 30 (1 month): 6 animals in Group 1 are euthanized, the sciatic nerves are exposed, imaged, and adhesions are assessed around the connector. The nerve segments (TREATMENT and Predicate CONTROL) are harvested for histopathologic analysis.

Day 90 (3 months): 6 animals in Group 3 are euthanized, the sciatic nerves are exposed, imaged, and adhesions are assessed around the connector. The nerve segments (TREATMENT and Predicate CONTROL) are harvested for histopathologic analysis.

### Study Outcomes:

Following surgery, all animals recovered without any adverse events. All animals showed mobility and movement in treated leg. In all cases, there was curling of toes. At Day 30 and Day 60, exposed treated site showed evidence of scar tissue and fibrotic adhesion around the Predicate CONTROL, however no fibrotic adhesion was seen around the CT wrap. The nerve with connector were cut out, imaged and cut into smaller samples for transverse histology analysis.

Explant Sections were processed for histology and slides stained with H&E (Hematoxylin & Eosin) and Trichrome. Further, immunostaining for nerve cells were performed to access reconnection of nerve in the gap.

### Example 3

A tissue conduit of the present invention was compared in vivo to a commercially available SIS product from Axogen. The process and use is shown in FIGS. 34, and 5. No scar tissue developed around the conduit of the present invention, but scar tissue developed around the Axogen product. Further, regarding the conduit of the present invention, no evidence of an immune response was seen, no evidence of foreign body response, evidence of new vascular cell production around the nerve, and evidence of interstitial cells within the conduit.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

While the invention has been described in some detail by way of illustration and example, it should be understood that the invention is susceptible to various modifications and alternative forms and is not restricted to the specific embodiments set forth in the Examples. It should be understood that these specific embodiments are not intended to limit the invention but, on the contrary, the intention is to cover all modifications, equivalents, and alternatives falling within the spirit and scope of the invention.

1-3. (canceled)

4. A nerve wrap comprising decellularized constructed tissue formed from extracellular matrix-producing cells combined with fibrinogen monomer and a polymerization catalyst, for nerve treatment.

5. The nerve wrap of claim 4 wherein the constructed tissue is also regenerative tissue.

6. The nerve wrap of claim 4 wherein the nerve wrap comprises a diameter and/or length that approximates the anatomy of one or more nerves selected from the group consisting of a CNS nerve, brachial plexus; radial nerve; ulnar nerve; median nerve.

7. A method of treating a nerve injury comprising treating the nerve with decellularized regenerative tissue formed from extracellular matrix-producing cells combined with fibrinogen monomer and a polymerization catalyst.

8. The method of claim 7 wherein treating comprises contacting or wrapping.

9. The method of claim 7 wherein the nerve injury includes carpel tunnel syndrome, nerve neuroma, and a transected nerve.

10. The method of claim 7 wherein the tissue is a nerve or one or more nerve ends.

11. (canceled)

12. The nerve wrap of claim 4 wherein the nerve wrap is tubular.

13. The nerve wrap of claim 12 wherein the tubular wrap comprises a slit.

14. A tissue communication product wherein one end of a damaged tissue and be in communication with a second end, wherein the product comprises a tube, tubular shape, or wrap comprised of extracellular-producing cells mixed with a fibrinogen monomer and a polymerization catalyst, and wherein said product includes a lumen or space that communicates between one end of a damaged tissue and another end.

15. The method of claim 14 wherein the tissue is a nerve or one or more nerve ends.

16. (canceled)
